(19) United States
(12) Patent Application Publication
(10) Pub. No.: US 2020/0202046 A1
(43) Pub. Date: Jun. 25, 2020

(54) Performance Prediction Method And System Of Pervious Concrete Based On Meso-Structure Reconstruction Model

(71) Applicant: Harbin Institute Of Technology, Shenzhen, Shenzhen City (CN)

(72) Inventors: Tiejun Liu, Shenzhen City (CN); Zhongzhen Wang, Shenzhen City (CN); Dujian Zou, Shenzhen City (CN); Mingming Shen, Shenzhen City (CN); Ao Zhou, Shenzhen City (CN)

(73) Assignee: Harbin Institute Of Technology, Shenzhen, Shenzhen City (CN)

(21) Appl. No.: 16/710,304

(22) Filed: Dec. 11, 2019

(30) Foreign Application Priority Data
Dec. 20, 2018 (CN) 201811591914.4

(51) Int. Cl. G06F 30/13; G06F 30/23; G01N 33/38; G01N 15/08

(52) U.S. Cl. CPC G06F 30/13; G06F 30/23; G06F 2111/10; G01N 15/08; G01N 33/383

(57) ABSTRACT

The present invention discloses a performance prediction method and system of pervious concrete based on a meso-structure reconstruction model. The method includes: obtaining a tomographic image of a coarse aggregate; extracting a coarse aggregate distribution region in the tomographic image of the coarse aggregate; calculating a maximum thickness of a coating; adding a cement-based coating with a preset thickness to the surface of the coarse aggregate in the coarse aggregate distribution region by a morphological operation method, to obtain a coating-added image; three-dimensionally reconstructing the coating-added image by a three-dimensional reconstruction method, to obtain a three-dimensional model of the pervious concrete; extracting a pore distribution region in the coating-added image; three-dimensionally reconstructing the pore distribution region by a three-dimensional reconstruction method, to obtain a three-dimensional model of a pore; and predicting a performance parameter of the pervious concrete corresponding to the three-dimensional model of the pervious concrete.

### TECHNICAL FIELD

The present invention relates to the technical field of pervious concrete performance prediction, and in particular, to a performance prediction method and system of pervious concrete based on a meso-structure reconstruction model.

### BACKGROUND

Pervious concrete consists of three parts: a coarse aggregate, a coarse aggregate cement-based coating and a pore. The performance of all aspects of the pervious concrete is directly dependent on the characteristics of the coarse aggregate and the coarse aggregate cement-based coating. The pervious concrete has a small amount of cement base and is often formed by pressure; thus, the coarse aggregate in its structure can be considered to be in a dense packing state. As the structure and performance of a selected coarse aggregate are known under the dense packing state, a pore structure of the coarse aggregate can be changed by artificially designing the performance and distribution of the cement base, thereby realizing model reconstruction and performance prediction of the pervious concrete.

At present, traditional methods use a volumetric method recommended in the national industry standard “Technical Specifications for Pervious Concrete Pavement” to perform the model reconstruction and performance prediction of the pervious concrete. This method can only predict the total porosity of the pervious concrete, and cannot predict pore characteristics, for example, a variety of pore characteristics such as pore size distribution, interconnected porosity, and porous channel tortuosity, and is more difficult to predict the perviousness performance and strength of the pervious concrete; moreover, model reconstruction based performance is largely deviated from actual performance, and is difficult to control. In addition, the traditional methods are only a rough summary of engineering experience, and are difficult to meet the refined design and prediction requirements of pervious concrete performance.

### SUMMARY

Based on this, the present invention provides a performance prediction method and system of pervious concrete based on a meso-structure reconstruction model, which can not only realize the prediction of a pore characteristic, but also predict perviousness performance and strength, and has high reliability.

To achieve the above purpose, the present invention provides the following technical solutions.

A performance prediction method of pervious concrete based on a meso-structure reconstruction model includes:

obtaining a tomographic image of a coarse aggregate, which is a tomographic image obtained by tomographically scanning the coarse aggregate in a dense packing state with an X-ray;

extracting a coarse aggregate distribution region in the tomographic image of the coarse aggregate;

calculating a maximum thickness of a coating, which is the maximum thickness of a cement-based coating coated on the surface of the coarse aggregate;

adding a cement-based coating with a preset thickness to the surface of the coarse aggregate in the coarse aggregate distribution region by a morphological operation method, to obtain a coating-added image, the preset thickness being smaller than the maximum thickness of the coating;

three-dimensionally reconstructing the coating-added image by a three-dimensional reconstruction method, to obtain a three-dimensional model of the pervious concrete;

extracting a pore distribution region in the coating-added image;

three-dimensionally reconstructing the pore distribution region by a three-dimensional reconstruction method, to obtain a three-dimensional model of a pore; and

predicting a performance parameter of the pervious concrete corresponding to the three-dimensional model of the pervious concrete, according to the three-dimensional model of the pervious concrete and the three-dimensional model of the pore, where the performance parameter includes a pore characteristic parameter, a perviousness coefficient, and strength; the pore characteristic parameter includes a total porosity, an interconnected porosity, pore parameter distribution, and porous channel tortuosity.

Preferably, the calculating a maximum thickness of a coating specifically includes:

three-dimensionally reconstructing the coarse aggregate distribution region by a three-dimensional reconstruction method, to obtain a three-dimensional model of the coarse aggregate;

obtaining a surface area of the coarse aggregate according to the three-dimensional model of the coarse aggregate;

obtaining the weight of an actual coarse aggregate corresponding to the tomographic image of the coarse aggregate, the weight of a coarse aggregate actually coated with the cement-based coating, and the density of a cement base; and

calculating the maximum thickness of the coating according to the surface area of the coarse aggregate, the weight of the actual coarse aggregate, the weight of the coarse aggregate actually coated with the cement-based coating, and the density of the cement base.

Preferably, the adding a cement-based coating with a preset thickness to the surface of the coarse aggregate in the coarse aggregate distribution region by a morphological operation method, to obtain a coating-added image specifically includes:

adding a cement-based coating with a preset thickness to an uncompressed area on the surface of the coarse aggregate by a pixel expansion algorithm, to obtain a first coating image, where the uncompressed area is an area in which a gap distance between the coarse aggregate and an adjacent coarse aggregate is greater than or equal to a preset distance; and

adding the cement-based coating with the preset thickness to a compressed area on the surface of the coarse aggregate by an image closing operation, to obtain a second coating image, where the compressed area is an area in which a gap distance between the coarse aggregate and an adjacent coarse aggregate is smaller than a preset distance, or is an overlapped position area of the coarse aggregate and the adjacent coarse aggregate, and the first coating image and the second coating image form the coating-added image.

Preferably, the calculating the maximum thickness of the coating according to the surface area of the coarse aggregate, the weight of the actual coarse aggregate, the weight of the coarse aggregate actually coated with the cement-based coating, and the density of the cement base is specifically:

$$MPT = \frac{{M}_{2} - {M}_{1}}{ρ \cdot S} ;$$

where, MPT represents the maximum thickness of the coating, S represents the surface area of the coarse aggregate, M₁ represents the weight of the actual coarse aggregate, M₂ represents the weight of the coarse aggregate actually coated with the cement-based coating, and ρ represents the density of the cement base.

Preferably, the predicting a performance parameter of the pervious concrete corresponding to the three-dimensional model of the pervious concrete, according to the three-dimensional model of the pervious concrete and the three-dimensional model of the pore specifically includes:

predicting a pore characteristic parameter of the pervious concrete corresponding to the three-dimensional model of the pervious concrete according to the three-dimensional model of the pore; and

predicting a perviousness performance parameter and strength of the pervious concrete corresponding to the three-dimensional model of the pervious concrete according to the three-dimensional model of the pervious concrete and the three-dimensional model of the pore.

Preferably, the predicting a pore characteristic parameter of the pervious concrete corresponding to the three-dimensional model of the pervious concrete according to the three-dimensional model of the pore specifically includes:

obtaining a total pore volume and an interconnected pore volume according to the three-dimensional model of the pore;

obtaining a total porosity according to the total pore volume and obtaining an interconnected porosity according to the interconnected pore volume;

extracting an edge contour of a pore pixel in the three-dimensional model of the pore;

calculating the area of each pore and the central axis of each porous channel according to the edge contour;

obtaining pore diameter distribution according to the area of each pore; and calculating the tortuosity of a porous channel according to the central axis of each porous channel;

$$τ = {\sum }_{i = 1}^{j} {l}_{i} / {\sum }_{i = 1}^{j} {H}_{i} ;$$

where, lᵢ represents the length of the central axis of an i-th porous channel, Hᵢ is a height difference of the central axis of the i-th porous channel, and j is total number of central axes of porous channels.

Preferably, the predicting a perviousness coefficient and strength of the pervious concrete corresponding to the three-dimensional model of the pervious concrete according to the three-dimensional model of the pervious concrete and the three-dimensional model of the pore specifically includes:

generating a finite element model according to the three-dimensional model of the pore;

calculating a pervious flow of the three-dimensional model of the pore in a unit time by the finite element model;

calculating the perviousness coefficient of the pervious concrete according to the three-dimensional model of the pervious concrete and the pervious flow

k=Q·L/A·Δh

where, Q represents the pervious flow, L represents a height of the three-dimensional model of the pervious concrete, and A represents a pervious cross-sectional area of an upper surface of the three-dimensional model of the pervious concrete, and Δh represents a pressure head of the upper surface of the three-dimensional model of the pervious concrete; and

calculating the strength of the pervious concrete according to the three-dimensional model of the pore

f_{PC}=f_{c}·(1−mϕ)·(dₐ/dₚ)ⁿ

where f_{c} represents the strength of the cement base; m and n are empirical coefficients, and both are integers; ϕ represents the total porosity; dₐ is a particle size of the coarse aggregate; dₚ represents an average pore diameter.

A performance prediction system of pervious concrete based on a meso-structure reconstruction model includes:

an image obtaining module, for obtaining a tomographic image of a coarse aggregate, which is a tomographic image obtained by tomographically scanning the coarse aggregate in a dense packing state with an X-ray;

a first extraction module, for extracting a coarse aggregate distribution region in the tomographic image of the coarse aggregate;

a calculation module, for calculating a maximum thickness of a coating, which is the maximum thickness of a cement-based coating coated on the surface of the coarse aggregate;

a coating adding module, for adding a cement-based coating with a preset thickness to the surface of the coarse aggregate in the coarse aggregate distribution region by a morphological operation method, to obtain a coating-added image, the preset thickness being smaller than the maximum thickness of the coating;

a first model reconstruction module, for three-dimensionally reconstructing the coating-added image by a three-dimensional reconstruction method, to obtain a three-dimensional model of the pervious concrete;

a second extraction module, for extracting a pore distribution region in the coating-added image;

a second model reconstruction module, for three-dimensionally reconstructing the pore distribution region by a three-dimensional reconstruction method, to obtain a three-dimensional model of a pore; and

a prediction module, for predicting a performance parameter of the pervious concrete corresponding to the three-dimensional model of the pervious concrete, according to the three-dimensional model of the pervious concrete and the three-dimensional model of the pore, where the performance parameter includes a pore characteristic parameter, a perviousness coefficient, and strength; the pore characteristic parameter includes a total porosity, an interconnected porosity, pore parameter distribution, and porous channel tortuosity.

Preferably, the calculation module specifically includes:

a model reconstruction unit, for three-dimensionally reconstructing the coarse aggregate distribution region by a three-dimensional reconstruction method, to obtain a three-dimensional model of the coarse aggregate;

a first obtaining unit, for obtaining a surface area of the coarse aggregate according to the three-dimensional model of the coarse aggregate;

a second obtaining unit, for obtaining the weight of an actual coarse aggregate corresponding to the tomographic image of the coarse aggregate, the weight of a coarse aggregate actually coated with the cement-based coating, and the density of the cement base; and

a calculation unit, for calculating the maximum thickness of the coating according to the surface area of the coarse aggregate, the weight of the actual coarse aggregate, the weight of the coarse aggregate actually coated with the cement-based coating, and the density of the cement base.

Preferably, the coating adding module specifically includes:

a first adding unit, for adding a cement-based coating with a preset thickness to an uncompressed area on the surface of the coarse aggregate by a pixel expansion algorithm, to obtain a first coating image, where the uncompressed area is an area in which a gap distance between the coarse aggregate and an adjacent coarse aggregate is greater than or equal to a preset distance; and

a second adding unit, for adding the cement-based coating with the preset thickness to a compressed area on the surface of the coarse aggregate by an image closing operation, to obtain a second coating image, where the compressed area is an area in which a gap distance between the coarse aggregate and an adjacent coarse aggregate is smaller than a preset distance, or is an overlapped position area of the coarse aggregate and the adjacent coarse aggregate, and the first coating image and the second coating image form the coating-added image.

Compared with the prior art, the present invention has the following beneficial effects.

The present invention provides a performance prediction method and system of pervious concrete based on a meso-structure reconstruction model, where the method includes: obtaining a tomographic image of a coarse aggregate; extracting a coarse aggregate distribution region in the tomographic image of the coarse aggregate; calculating a maximum thickness of a coating; adding a cement-based coating with a preset thickness to the surface of the coarse aggregate in the coarse aggregate distribution region by a morphological operation method, to obtain a coating-added image, where the preset thickness is smaller than the maximum thickness of the coating; three-dimensionally reconstructing the coating-added image by a three-dimensional reconstruction method, to obtain a three-dimensional model of the pervious concrete; extracting a pore distribution region in the coating-added image; three-dimensionally reconstructing the pore distribution region by a three-dimensional reconstruction method, to obtain a three-dimensional model of a pore; and predicting a performance parameter of the pervious concrete corresponding to the three-dimensional model of the pervious concrete, according to the three-dimensional model of the pervious concrete and the three-dimensional model of the pore. The present invention can not only realize the prediction of a pore characteristic, but also predict perviousness performance and strength, and has high reliability, and provides a method basis for the actual design and application of the pervious concrete, thereby realizing the refined design and prediction of the performance of the pervious concrete.

### BRIEF DESCRIPTION OF THE DRAWINGS

To describe the technical solutions in the embodiments of the present invention or in the prior art more clearly, the following briefly introduces the accompanying drawings required for describing the embodiments. Apparently, the accompanying drawings in the following description show merely some embodiments of the present invention, and a person of ordinary skill in the art may still derive other drawings from these accompanying drawings without creative efforts.

FIG. 1 is a flow diagram of a performance prediction method of pervious concrete based on a meso-structure reconstruction model according to an embodiment of the present invention;

FIG. 2 is a schematic structural diagram of a tomographic image of a coarse aggregate and a three-dimensional model of the coarse aggregate according to an embodiment of the present invention;

FIG. 3 is a schematic diagram of an uncompressed area and a compressed area;

FIG. 4 is a schematic structural diagram of a model of an uncompressed area and a compressed area added with a cement-based coating;

FIG. 5 is a schematic structural diagram of a coating-added image and a three-dimensional model of pervious concrete according to an embodiment of the present invention;

FIG. 6 is a schematic structural diagram of a three-dimensional model of a pore according to an embodiment of the present invention;

FIG. 7 is a schematic structural diagram of an edge contour and a central axis of a porous channel according to an embodiment of the present invention;

FIG. 8 is a schematic structural diagram of a finite element model according to an embodiment of the present invention; and

FIG. 9 is a schematic structural diagram of a performance prediction system of pervious concrete based on a meso-structure reconstruction model according to an embodiment of the present invention.

### DETAILED DESCRIPTION

The following clearly and completely describes the technical solutions in the embodiments of the present invention with reference to the accompanying drawings in the embodiments of the present invention. Apparently, the described embodiments are merely a part rather than all of the embodiments of the present invention. All other embodiments obtained by a person of ordinary skill in the art based on the embodiments of the present invention without creative efforts shall fall within the protection scope of the present invention.

To make the foregoing objectives, features, and advantages of the present invention more comprehensible, the following describes the present invention in more detail with reference to accompanying drawings and specific implementations.

FIG. 1 is a flow diagram of a performance prediction method of pervious concrete based on a meso-structure reconstruction model according to an embodiment of the present invention.

Referring to FIG. 1, the performance prediction method of pervious concrete based on a meso-structure reconstruction model according to the embodiment includes the following steps.

Step S1: obtain a tomographic image of a coarse aggregate.

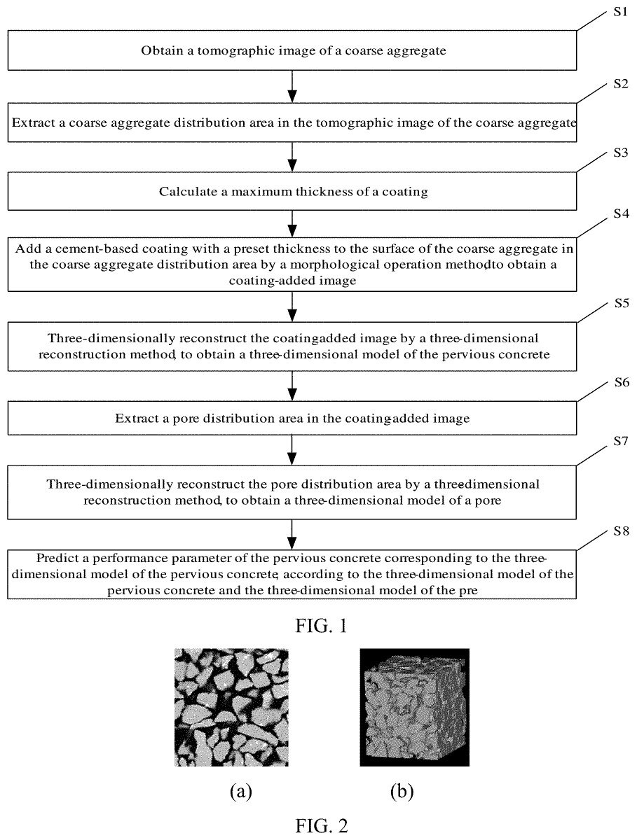

The tomographic image of the coarse aggregate is a tomographic image obtained by tomographically scanning the coarse aggregate in a dense packing state with an X-ray. In order to improve the accuracy of the tomographic image of the coarse aggregate, the resolution of the image can be enlarged. The tomographic image of the coarse aggregate is shown in part (a) of FIG. 2.

Step S2: extract a coarse aggregate distribution region in the tomographic image of the coarse aggregate.

Specifically, the coarse aggregate is distinguished from a pore by an image gray value to obtain the coarse aggregate distribution region.

Step S3: calculate a maximum thickness of a coating. The maximum thickness of the coating is the maximum thickness of a cement-based coating coated on the surface of the coarse aggregate.

The step S3 specifically includes the following process.

Three-dimensionally reconstruct the coarse aggregate distribution region by a three-dimensional reconstruction method, to obtain a three-dimensional model of the coarse aggregate. In this embodiment, the three-dimensional model of the coarse aggregate is obtained by three-dimensional reconstruction by Materiaise's interactive medical image control system (MIMICS) software. The three-dimensional model of the coarse aggregate is shown in part (b) of FIG. 2.

Obtain a surface area of the coarse aggregate according to the three-dimensional model of the coarse aggregate.

Obtain the weight of an actual coarse aggregate corresponding to the tomographic image of the coarse aggregate, the weight of a coarse aggregate actually coated with the cement-based coating, and the density of a cement base. The weight of the actual coarse aggregate is determined according to the tomographic image of the coarse aggregate; the weight of the coarse aggregate actually coated with the cement-based coating is determined by testing, specifically: evenly stir an actual coarse aggregate having a known weight with a cement slurry having a known proportion, place the coarse aggregate coated with the cement slurry on a screen, vibrate the screen to remove a cement slurry that is not stably coated on the surface of the coarse aggregate to obtain a coarse aggregate coated with the cement-based coating, and weight to obtain the weight of the coarse aggregate actually coated with the cement-based coating.

Calculate the maximum thickness of the coating according to the surface area of the coarse aggregate, the weight of the actual coarse aggregate, the weight of the coarse aggregate actually coated with the cement-based coating, and the density of the cement base, the maximum thickness of the coating being

$$MPT = \frac{{M}_{2} - {M}_{1}}{ρ \cdot S} ;$$

where, MPT represents the maximum thickness of the coating, S represents the surface area of the coarse aggregate, M₁ represents the weight of the actual coarse aggregate, M₂ represents the weight of the coarse aggregate actually coated with the cement-based coating, and ρ represents the density of the cement base.

Step S4: add a cement-based coating with a preset thickness to the surface of the coarse aggregate in the coarse aggregate distribution region by a morphological operation method, to obtain a coating-added image. The preset thickness is smaller than the maximum thickness of the coating.

The step S4 is specifically as follows.

(1) Use a pixel expansion algorithm, select an uncompressed area on the surface of the coarse aggregate as a negative plate for pixel expansion, expand an edge of the coarse aggregate outwards, and add a cement-based coating with a preset thickness to the uncompressed area on the surface of the coarse aggregate, to obtain a first coating image, where the uncompressed area is an area in which a gap distance between the coarse aggregate and an adjacent coarse aggregate is greater than or equal to a preset distance.

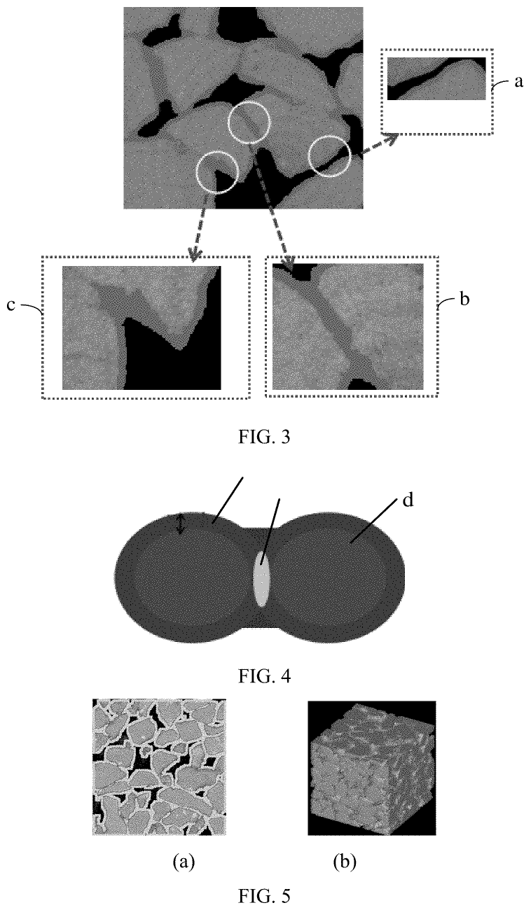

(2) Add the cement-based coating with the preset thickness to a compressed area on the surface of the coarse aggregate by an image closing operation, to obtain a second coating image, where the compressed area is an area in which a gap distance between the coarse aggregate and an adjacent coarse aggregate is smaller than a preset distance, or is an overlapped position area of the coarse aggregate and the adjacent coarse aggregate. FIG. 3 is a schematic diagram of the uncompressed area and the compressed area. In the figure, a is the area in which the gap distance between the coarse aggregate and the adjacent coarse aggregate is greater than or equal to the preset distance; b is the area in which the gap distance between the coarse aggregate and the adjacent coarse aggregate is smaller than the preset distance; and c is the overlapped position area of the coarse aggregate and the adjacent coarse aggregate. FIG. 4 is a schematic structural diagram of a model of the uncompressed area and the compressed area added with the cement-based coating. In the figure, V₁ represents the uncompressed area, V₁ represents the compressed area, d represents the coarse aggregate, and a double sided arrow represents the thickness of the cement-based coating. The first coating image and the second coating image form the coating-added image, and the coating-added image is shown in part (a) of FIG. 5.

Step S5: three-dimensionally reconstruct the coating-added image by a three-dimensional reconstruction method, to obtain a three-dimensional model of the pervious concrete. In this embodiment, the three-dimensional model of the pervious concrete is obtained by three-dimensional reconstruction by the MIMICS software. The three-dimensional model of the pervious concrete is shown in part (b) of FIG. 5.

Step S6: extract a pore distribution region in the coating-added image.

Specifically:

Subtract a cement base area and a coarse aggregate area from the coating-added image by a Boolean operation, to obtain the pore distribution region.

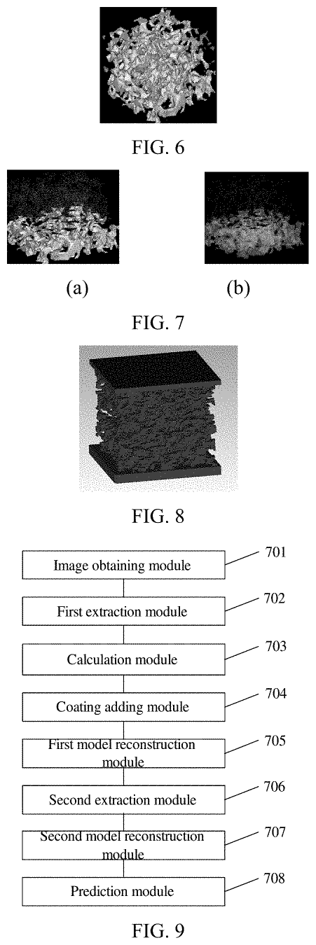

Step S7: three-dimensionally reconstruct the pore distribution region by a three-dimensional reconstruction method, to obtain a three-dimensional model of a pore. In this embodiment, the three-dimensional model of the pore is obtained by three-dimensional reconstruction by the MIMICS software. The three-dimensional model of the pore is shown in FIG. 6.

Step S8: predict a performance parameter of the pervious concrete corresponding to the three-dimensional model of the pervious concrete, according to the three-dimensional model of the pervious concrete and the three-dimensional model of the pore. The performance parameter includes a pore characteristic parameter, a perviousness coefficient, and strength; the pore characteristic parameter includes a total porosity, an interconnected porosity, pore parameter distribution, and porous channel tortuosity.

The step specifically includes the following process.

(1) Predict a pore characteristic parameter of the pervious concrete corresponding to the three-dimensional model of the pervious concrete according to the three-dimensional model of the pore. Specifically:

obtain a total pore volume and an interconnected pore volume according to the three-dimensional model of the pore; specifically, if the volume of a single volume pixel is known, cumulatively calculate the number of volume pixels of the three-dimensional model of the pore to obtain the total pore volume, then determine the connectivity of the volume pixels of the three-dimensional model of the pore to extract connected and unconnected parts, and obtain the interconnected pore volume;

obtain a total porosity according to the total pore volume and obtain an interconnected porosity according to the interconnected pore volume;

automatically extract an edge contour of a pore pixel in the three-dimensional model of the pore by the MIMICS software, the edge contour being shown in part (a) of FIG. 7;

calculate the area of each pore according to the edge contour, and convert the area into a diameter of an equal area circle, to obtain pore diameter distribution; and

calculate a center of the edge contour, use a quadratic smooth curve and connect a center of an adjacent interconnected pore to obtain a central axis of a porous channel, extract a length and a height difference of the central axis, and calculate the tortuosity of the porous channel by the following formula

$$τ = {\sum }_{i - 1}^{j} {l}_{i} / {\sum }_{i - 1}^{j} {H}_{i} ;$$

where, lᵢ represents the length of the central axis of an i-th porous channel, Hᵢ is the height difference of the central axis of the i-th porous channel, j is a total number of central axes of porous channels, and the central axis of the porous channel is shown in part (b) of FIG. 7.

(2) Predict a perviousness performance parameter and strength of the pervious concrete corresponding to the three-dimensional model of the pervious concrete according to the three-dimensional model of the pervious concrete and the three-dimensional model of the pore. Specifically:

generate a finite element model of a hexahedral mesh by the three-dimensional model of the pore by the MIMICS software, where the finite element model is shown in FIG. 8;

introduce the finite element model into fluid simulation software Fluent_3D, and calculate a pervious flow of the three-dimensional model of the pore in a unit time;

calculate the perviousness coefficient of the pervious concrete according to the three-dimensional model of the pervious concrete and the pervious flow

k=Q·L/A·Δh

where, Q represents the pervious flow, L represents a height of the three-dimensional model of the pervious concrete, and A represents a pervious cross-sectional area of an upper surface of the three-dimensional model of the pervious concrete, and Δh represents a pressure head of the upper surface of the three-dimensional model of the pervious concrete; and

calculate the strength of the pervious concrete according to the three-dimensional model of the pore

f_{PC}=f_{c}·(1−mϕ)·(dₐ/dₚ)ⁿ

where f_{c} represents the strength of the cement base; m and n are empirical coefficients, and both are integers; ϕ represents the total porosity; dₐ is a particle size of the coarse aggregate; dₚ represents an average pore diameter.

The performance prediction method of pervious concrete based on a meso-structure reconstruction model in the present embodiment fills a gap in such fields as the pore structure design, perviousness performance design, strength design and performance prediction of the pervious concrete, and can not only realize the prediction of a pore characteristic, but also predict perviousness performance and strength, and has high reliability, and provides a method basis for the actual design and application of the pervious concrete, thereby realizing the refined design and prediction of the performance of the pervious concrete.

The present invention provides a performance prediction system of pervious concrete based on a meso-structure reconstruction model. FIG. 9 is a schematic structural diagram of the performance prediction system of pervious concrete based on a meso-structure reconstruction model according to an embodiment of the present invention. The system includes:

an image obtaining module 701, for obtaining a tomographic image of a coarse aggregate, which is a tomographic image obtained by tomographically scanning the coarse aggregate in a dense packing state with an X-ray;

a first extraction module 702, for extracting a coarse aggregate distribution region in the tomographic image of the coarse aggregate;

a calculation module 703, for calculating a maximum thickness of a coating, which is the maximum thickness of a cement-based coating coated on the surface of the coarse aggregate;

a coating adding module 704, for adding a cement-based coating with a preset thickness to the surface of the coarse aggregate in the coarse aggregate distribution region by a morphological operation method, to obtain a coating-added image, the preset thickness being smaller than the maximum thickness of the coating;

a first model reconstruction module 705, for three-dimensionally reconstructing the coating-added image by a three-dimensional reconstruction method, to obtain a three-dimensional model of the pervious concrete;

a second extraction module 706, for extracting a pore distribution region in the coating-added image;

a second model reconstruction module 707, for three-dimensionally reconstructing the pore distribution region by a three-dimensional reconstruction method, to obtain a three-dimensional model of a pore; and

a prediction module 708, for predicting a performance parameter of the pervious concrete corresponding to the three-dimensional model of the pervious concrete, according to the three-dimensional model of the pervious concrete and the three-dimensional model of the pore, where the performance parameter includes a pore characteristic parameter, a perviousness coefficient, and strength; the pore characteristic parameter includes a total porosity, an interconnected porosity, pore parameter distribution, and porous channel tortuosity.

As an optional implementation, the calculation module 703 specifically includes:

a model reconstruction unit, for three-dimensionally reconstructing the coarse aggregate distribution region by a three-dimensional reconstruction method, to obtain a three-dimensional model of the coarse aggregate;

a first obtaining unit, for obtaining a surface area of the coarse aggregate according to the three-dimensional model of the coarse aggregate;

a second obtaining unit, for obtaining the weight of an actual coarse aggregate corresponding to the tomographic image of the coarse aggregate, the weight of a coarse aggregate actually coated with the cement-based coating, and the density of the cement base; and

a calculation unit, for calculating the maximum thickness of the coating according to the surface area of the coarse aggregate, the weight of the actual coarse aggregate, the weight of the coarse aggregate actually coated with the cement-based coating, and the density of the cement base.

As an optional implementation, the coating adding module 704 specifically includes:

a first adding unit, for adding a cement-based coating with a preset thickness to an uncompressed area on the surface of the coarse aggregate by a pixel expansion algorithm, to obtain a first coating image, where the uncompressed area is an area in which a gap distance between the coarse aggregate and an adjacent coarse aggregate is greater than or equal to a preset distance; and

a second adding unit, for adding the cement-based coating with the preset thickness to a compressed area on the surface of the coarse aggregate by an image closing operation, to obtain a second coating image, where the compressed area is an area in which a gap distance between the coarse aggregate and an adjacent coarse aggregate is smaller than a preset distance, or is an overlapped position area of the coarse aggregate and the adjacent coarse aggregate, and the first coating image and the second coating image form the coating-added image.

As an optional implementation, the prediction module 708 specifically includes:

a first prediction unit, for predicting a pore characteristic parameter of the pervious concrete corresponding to the three-dimensional model of the pervious concrete according to the three-dimensional model of the pore; and

a second prediction unit, for predicting a perviousness performance parameter and strength of the pervious concrete corresponding to the three-dimensional model of the pervious concrete according to the three-dimensional model of the pervious concrete and the three-dimensional model of the pore.

As an optional implementation, the first prediction unit specifically includes:

a volume obtaining subunit, for obtaining a total pore volume and an interconnected pore volume according to the three-dimensional model of the pore;

a first parameter prediction subunit, for obtaining a total porosity according to the total pore volume and obtaining an interconnected porosity according to the interconnected pore volume;

an extraction subunit, for extracting an edge contour of a pore pixel in the three-dimensional model of the pore;

a first calculation subunit, for calculating the area of each pore and the central axis of each porous channel according to the edge contour;

a second parameter prediction subunit, for obtaining pore diameter distribution according to the area of each pore; and

a third parameter prediction subunit, for calculating the tortuosity of a porous channel according to the central axis of each porous channel;

$$τ = {\sum }_{i = 1}^{j} {l}_{i} / {\sum }_{i = 1}^{j} {H}_{i} ;$$

where, lᵢ represents the length of the central axis of an i-th porous channel, Hᵢ is a height difference of the central axis of the i-th porous channel, and j is total number of central axes of porous channels.

The second prediction unit specifically includes:

a model generation subunit, for generating a finite element model according to the three-dimensional model of the pore;

a second calculation subunit, for calculating a pervious flow of the three-dimensional model of the pore in a unit time by the finite element model;

a fourth parameter prediction subunit, for calculating the perviousness coefficient of the pervious concrete according to the three-dimensional model of the pervious concrete and the pervious flow

k=Q·L/A·Δh

where, Q represents the pervious flow, L represents a height of the three-dimensional model of the pervious concrete, and A represents a pervious cross-sectional area of an upper surface of the three-dimensional model of the pervious concrete, and Δh represents a pressure head of the upper surface of the three-dimensional model of the pervious concrete; and

a fifth parameter prediction subunit, for calculating the strength of the pervious concrete according to the three-dimensional model of the pore

f_{PC}=f_{c}·(1−mϕ)·(dₐ/dₚ)ⁿ

where f_{c} represents the strength of the cement base; m and n are empirical coefficients, and both are integers; ϕ represents the total porosity; dₐ is a particle size of the coarse aggregate; dₚ represents an average pore diameter.

The performance prediction system of pervious concrete based on a meso-structure reconstruction model in the present embodiment can not only realize the prediction of a pore characteristic, but also predict perviousness performance and strength, and has high reliability.

For a system disclosed in the embodiments, since it corresponds to the method disclosed in the embodiments, the description is relatively simple, and reference can be made to the method description.

Several examples are used for illustration of the principles and implementation manners of the present invention. The description of the embodiments is used to help illustrate the method of the present invention and its core principles. In addition, those skilled in the art can make various modifications in terms of specific manners and scope of application in accordance with the teachings of the present invention. In conclusion, the content of this specification shall not be construed as a limitation to the present invention.

What is claimed is:

1. A performance prediction method of pervious concrete based on a meso-structure reconstruction model, comprising:
obtaining a tomographic image of a coarse aggregate, which is a tomographic image obtained by tomographically scanning the coarse aggregate in a dense packing state with an X-ray;
extracting a coarse aggregate distribution region in the tomographic image of the coarse aggregate;
calculating a maximum thickness of a coating, which is the maximum thickness of a cement-based coating coated on the surface of the coarse aggregate;
adding a cement-based coating with a preset thickness to the surface of the coarse aggregate in the coarse aggregate distribution region by a morphological operation method, to obtain a coating-added image, the preset thickness being smaller than the maximum thickness of the coating;
three-dimensionally reconstructing the coating-added image by a three-dimensional reconstruction method, to obtain a three-dimensional model of the pervious concrete;
extracting a pore distribution region in the coating-added image;
three-dimensionally reconstructing the pore distribution region by a three-dimensional reconstruction method, to obtain a three-dimensional model of a pore; and
predicting a performance parameter of the pervious concrete corresponding to the three-dimensional model of the pervious concrete, according to the three-dimensional model of the pervious concrete and the three-dimensional model of the pore, wherein the performance parameter comprises a pore characteristic parameter, a perviousness coefficient, and strength; the pore characteristic parameter comprises a total porosity, an interconnected porosity, pore parameter distribution, and porous channel tortuosity.

2. The performance prediction method of pervious concrete based on a meso-structure reconstruction model according to claim 1, wherein the calculating a maximum thickness of a coating specifically comprises:
three-dimensionally reconstructing the coarse aggregate distribution region by a three-dimensional reconstruction method, to obtain a three-dimensional model of the coarse aggregate;
obtaining a surface area of the coarse aggregate according to the three-dimensional model of the coarse aggregate;
obtaining the weight of an actual coarse aggregate corresponding to the tomographic image of the coarse aggregate, the weight of a coarse aggregate actually coated with the cement-based coating, and the density of a cement base; and
calculating the maximum thickness of the coating according to the surface area of the coarse aggregate, the weight of the actual coarse aggregate, the weight of the coarse aggregate actually coated with the cement-based coating, and the density of the cement base.

3. The performance prediction method of pervious concrete based on a meso-structure reconstruction model according to claim 1, wherein the adding a cement-based coating with a preset thickness to the surface of the coarse aggregate in the coarse aggregate distribution region by a morphological operation method, to obtain a coating-added image specifically comprises:
adding a cement-based coating with a preset thickness to an uncompressed area on the surface of the coarse aggregate by a pixel expansion algorithm, to obtain a first coating image, wherein the uncompressed area is an area in which a gap distance between the coarse aggregate and an adjacent coarse aggregate is greater than or equal to a preset distance; and
adding the cement-based coating with the preset thickness to a compressed area on the surface of the coarse aggregate by an image closing operation, to obtain a second coating image, wherein the compressed area is an area in which a gap distance between the coarse aggregate and an adjacent coarse aggregate is smaller than a preset distance, or is an overlapped position area of the coarse aggregate and the adjacent coarse aggregate, and the first coating image and the second coating image form the coating-added image.

4. The performance prediction method of pervious concrete based on a meso-structure reconstruction model according to claim 2, wherein the calculating the maximum thickness of the coating according to the surface area of the coarse aggregate, the weight of the actual coarse aggregate, the weight of the coarse aggregate actually coated with the cement-based coating, and the density of the cement base is specifically:
$$MPT = \frac{{M}_{2} - {M}_{1}}{ρ \cdot S} ;$$
wherein, MPT represents the maximum thickness of the coating, S represents the surface area of the coarse aggregate, M₁ represents the weight of the actual coarse aggregate, M₂ represents the weight of the coarse aggregate actually coated with the cement-based coating, and ρ represents the density of the cement base.

5. The performance prediction method of pervious concrete based on a meso-structure reconstruction model according to claim 1, wherein the predicting a performance parameter of the pervious concrete corresponding to the three-dimensional model of the pervious concrete, according to the three-dimensional model of the pervious concrete and the three-dimensional model of the pore specifically comprises:
predicting a pore characteristic parameter of the pervious concrete corresponding to the three-dimensional model of the pervious concrete according to the three-dimensional model of the pore; and
predicting a perviousness performance parameter and strength of the pervious concrete corresponding to the three-dimensional model of the pervious concrete according to the three-dimensional model of the pervious concrete and the three-dimensional model of the pore.

6. The performance prediction method of pervious concrete based on a meso-structure reconstruction model according to claim 5, wherein the predicting a pore characteristic parameter of the pervious concrete corresponding to the three-dimensional model of the pervious concrete according to the three-dimensional model of the pore specifically comprises:
obtaining a total pore volume and an interconnected pore volume according to the three-dimensional model of the pore;
obtaining a total porosity according to the total pore volume and obtaining an interconnected porosity according to the interconnected pore volume;
extracting an edge contour of a pore pixel in the three-dimensional model of the pore;
calculating the area of each pore and the central axis of each porous channel according to the edge contour;
obtaining pore diameter distribution according to the area of each pore; and
calculating the tortuosity of a porous channel according to the central axis of each porous channel;
$$τ = {\sum }_{i = 1}^{j} {l}_{i} / {\sum }_{i = 1}^{j} {H}_{i} ;$$
wherein, lᵢ represents the length of the central axis of an i-th porous channel, Hᵢ is a height difference of the central axis of the i-th porous channel, and j is total number of central axes of porous channels.

7. The performance prediction method of pervious concrete based on a meso-structure reconstruction model according to claim 5, wherein the predicting a perviousness coefficient and strength of the pervious concrete corresponding to the three-dimensional model of the pervious concrete according to the three-dimensional model of the pervious concrete and the three-dimensional model of the pore specifically comprises:
generating a finite element model according to the three-dimensional model of the pore;
calculating a pervious flow of the three-dimensional model of the pore in a unit time by the finite element model;
calculating the perviousness coefficient of the pervious concrete according to the three-dimensional model of the pervious concrete and the pervious flow
k=Q·L/A·Δh
wherein, Q represents the pervious flow, L represents a height of the three-dimensional model of the pervious concrete, and A represents a pervious cross-sectional area of an upper surface of the three-dimensional model of the pervious concrete, and Δh represents a pressure head of the upper surface of the three-dimensional model of the pervious concrete; and
calculating the strength of the pervious concrete according to the three-dimensional model of the pore
f_{PC}=f_{c}·(1−mϕ)·(dₐ/dₚ)ⁿ
wherein f_{c} represents the strength of the cement base; m and n are empirical coefficients, and both are integers; ϕ represents the total porosity; dₐ is a particle size of the coarse aggregate; dₚ represents an average pore diameter.

8. A performance prediction system of pervious concrete based on a meso-structure reconstruction model, comprising:
an image obtaining module, for obtaining a tomographic image of a coarse aggregate, which is a tomographic image obtained by tomographically scanning the coarse aggregate in a dense packing state with an X-ray;
a first extraction module, for extracting a coarse aggregate distribution region in the tomographic image of the coarse aggregate;
a calculation module, for calculating a maximum thickness of a coating, which is the maximum thickness of a cement-based coating coated on the surface of the coarse aggregate;
a coating adding module, for adding a cement-based coating with a preset thickness to the surface of the coarse aggregate in the coarse aggregate distribution region by a morphological operation method, to obtain a coating-added image, the preset thickness being smaller than the maximum thickness of the coating;
a first model reconstruction module, for three-dimensionally reconstructing the coating-added image by a three-dimensional reconstruction method, to obtain a three-dimensional model of the pervious concrete;
a second extraction module, for extracting a pore distribution region in the coating-added image;
a second model reconstruction module, for three-dimensionally reconstructing the pore distribution region by a three-dimensional reconstruction method, to obtain a three-dimensional model of a pore; and
a prediction module, for predicting a performance parameter of the pervious concrete corresponding to the three-dimensional model of the pervious concrete, according to the three-dimensional model of the pervious concrete and the three-dimensional model of the pore, wherein the performance parameter comprises a pore characteristic parameter, a perviousness coefficient, and strength; the pore characteristic parameter comprises a total porosity, an interconnected porosity, pore parameter distribution, and porous channel tortuosity.

9. The performance prediction system of pervious concrete based on a meso-structure reconstruction model according to claim 8, wherein the calculation module specifically comprises:
a model reconstruction unit, for three-dimensionally reconstructing the coarse aggregate distribution region by a three-dimensional reconstruction method, to obtain a three-dimensional model of the coarse aggregate;
a first obtaining unit, for obtaining a surface area of the coarse aggregate according to the three-dimensional model of the coarse aggregate;
a second obtaining unit, for obtaining the weight of an actual coarse aggregate corresponding to the tomographic image of the coarse aggregate, the weight of a coarse aggregate actually coated with the cement-based coating, and the density of the cement base; and
a calculation unit, for calculating the maximum thickness of the coating according to the surface area of the coarse aggregate, the weight of the actual coarse aggregate, the weight of the coarse aggregate actually coated with the cement-based coating, and the density of the cement base.

10. The performance prediction system of pervious concrete based on a meso-structure reconstruction model according to claim 8, wherein the coating adding module specifically comprises:
a first adding unit, for adding a cement-based coating with a preset thickness to an uncompressed area on the surface of the coarse aggregate by a pixel expansion algorithm, to obtain a first coating image, wherein the uncompressed area is an area in which a gap distance between the coarse aggregate and an adjacent coarse aggregate is greater than or equal to a preset distance; and
a second adding unit, for adding the cement-based coating with the preset thickness to a compressed area on the surface of the coarse aggregate by an image closing operation, to obtain a second coating image, wherein the compressed area is an area in which a gap distance between the coarse aggregate and an adjacent coarse aggregate is smaller than a preset distance, or is an overlapped position area of the coarse aggregate and the adjacent coarse aggregate, and the first coating image and the second coating image form the coating-added image.
